(19) United States
(12) Patent Application Publication
(10) Pub. No.: US 2003/0091533 A1
(43) Pub. Date: May 15, 2003

(54) Regulation of mononuclear phagocyte stimulation

(76) Inventors: Jacques Van Snick, Brussels (BE); Jean-Christophe Renauld, Brussels (BE); Youssef Ouadrhiri, Brussels (BE); Charles Pilette, Achet (BE)

Correspondence Address: FULBRIGHT & JAWORSKI L.L.P., Market Square, 801 Pennsylvania Avenue, N.W., Washington, DC, 20004-2615, US

(21) Appl. No.: 10/272,912

(22) Filed: Oct. 18, 2002

Related U.S. Application Data
(60) Provisional application No. 60/330,084, filed on Oct. 19, 2001.

(51) Int. Cl. A61K 38/20

(52) U.S. Cl. 424/85.2

(57) ABSTRACT

This invention relates to methods for inhibiting the production of reactive oxygen intermediates and to methods for altering the production of cytokines by stimulated mononuclear phagocytes, e.g., peripheral blood monocytes or alveolar macrophages. The invention also relates to methods for treating a subject with a pathologic condition, or at risk for developing a pathologic condition, associated with stimulated mononuclear phagocytes by administering IL-9 to the subject prior to mononuclear phagocyte stimulation.

### FIELD OF THE INVENTION

This application relates to methods for regulating oxidative burst and cytokine release by stimulated mononuclear phagocytes. This invention also relates to methods for treating a pathologic disorder associated with stimulated mononuclear phagocytes comprising administering an effective amount of IL-9 to a patient having the disorder, or a subject at risk of developing the disorder, for a sufficient time to inhibit or prevent stimulation of mononuclear phagocytes. The methods also relate to preventing or inhibiting tissue injury in a subject at risk for tissue damage from stimulated mononuclear phagocytes and to preventing or inhibiting the onset and progression of sepsis in a subject in need thereof comprising administering an effective amount of IL-9 to the subject for a sufficient time to inhibit or prevent stimulation of mononuclear phagocytes.

### BACKGROUND OF THE INVENTION

IL-9 is considered to be a Th2 cytokine that is inducible by both IL-4 dependent and IL-4 independent pathways, based on its restricted production by Th2 clones in vitro as well as its expression in Th2 type responses in vivo (see e.g., Gessner et al., Immunobiology, 189:419 (1993); Svetic et al., J. Immunol., 150:3434 (1993); Faulkner et al., Infect. Immunol., 66:3832 (1998); Kopf et al., Nature 362:245 (1993), and; Monteyne et al., J. Immunol. 159:2616 (1997)). Studies have shown that IL-9 has both beneficial and deleterious effects. For example, IL-9 has been implicated in host inflammatory processes, enhancing production of IgE and IgG (U.S. Pat. No. 5,132,109 and 5,246,701); in modulating cell apoptosis (U.S. Pat. No. 5,824,551); in the treatment of autoimmune disorders (U.S. Pat. No. 5,830,454), and; in the treatment of interstitial lung disease (U.S. Pat. No. 5,935,929). In vivo, IL-9 was shown to protect naive mice against various parasitic infections such as Trichuris muris, possibly through induction of blood hypereosinophilia (Richard et al., Proc. Natl Acad. Sci. USA 97: 767-772 (2000)). Prophylactic administration of IL-9 also protects mice from death in a model of sepsis induced by intravenous injection of LPS or Pseudomonas aeruginosa (Grohmann et al., J. Immunol. 164: 4197-4203 (2000)). This protective effect, also observed with IL-4, was associated with a strong reduction of serum levels of TNF-α, IL-12/p40, and IFN-γ, and with a dramatic increase of IL-10. In addition, in a silica-induced lung fibrosis model, IL-9 had a beneficial anti-fibrotic effect associated with an inhibition of the silica-induced up regulation of IL-4 expression (Arras et al., Am. J. Respir. Cell Molec. Biol. Am J. Respir. Cell. Molec. Biol. 24:368-375 (2001)).

In contrast to the beneficial effects of IL-9 described above, IL-9 transgenic mouse models and genetic studies indicate an important deleterious role for this cytokine in the pathogenesis of chronic asthma. Mice overexpressing IL-9 present characteristics mimicking the human disease, such as airway infiltration by mast cells (Godfraind et al., J. Immunol., 160: 3989-3996 (1998)) and eosinophils, as well as bronchial hyperresponsiveness. Mice overexpressing IL-9 selectively in their airways displayed airway infiltration by B and T lymphocytes and possibly macrophages. This is in addition to bronchial remodeling, characterized by epithelial mucoid hypertrophy and subepithelial fibrosis, which are associated with increased airway resistance (Temann et al., J. Exp. Med. 188: 1307-1320 (1998). It has been suggested that the genetic linkage between increased total serum IgE, a major asthma-related phenotypic feature, and the human 5q31 cytokine cluster gene locus involves the IL-9 gene (Nicolaides et al., Proc. Natl. Acad. Sci. USA, 94:13175-13180 (1997)). IL-9 has also been identified as a crucial factor mediating the up regulation of mucin gene transcription observed in airway epithelial cells from asthmatic subjects (Longphre et al., J. Clin. Invest., 104: 1375-1382 (1999) and IL-9-deficient mice are characterized by a defect in mast cell and mucus production in the lung (Townsend et al., Immunity, 13: 573-583 (2000)). It has also been suggested that IL-9 is involved in T-cell oncogenesis because IL-9 transgenic mice display an increased incidence of thymic lymphomas, (Renauld and Van Snick, Interleukin-9. In The Cytokine Handbook. A. Thomson, editor. Third edition, Academic Press, Chapter 11, pp313-331(1998)). In addition, IL-9 is implicated in the initiation and progression of atherosclerotic plaques in mice, as suggested in co-pending U.S. provisional application No. 60/284,232 filed Apr. 18, 2001 incorporated herein by reference.

Mononuclear phagocyte stimulation is associated with various pathologic conditions, e.g., inflammatory and autoimmune diseases, such as, sepsis, asthma inflammatory bowel diseases, e.g., ulcerative colitis and Crohn's disease, and tissue damage, e.g., damage to articular tissue (arthritis), liver tissue, lung tissue and vascular tissue. In a mouse model of acute lung injury induced by immune complexes, reactive oxygen intermediates (ROI), e.g., 02- and H₂O₂, have been shown to mediate tissue damage, and, moreover, both IL-4 and IL-10 exert a beneficial effect on this disorder (Mulligan et al., J Immunol., 1993; 151: 5666-5674). Oxygen radicals may play a role in active episodes of small-intestinal ischemia, ulcerative colitis, pancreatitis and gastric ulcer (Otamiri and Sjodahl, Dig. Dis., 9 (3):133-41 (1991)), and the production of oxygen radicals by macrophages in response to LPS is increased in subjects with inflammatory bowel disease. There is a large population of macrophages in the normal intestinal mucosa and studies indicate that the normal intestinal macrophages are not easily induced to mediate acute inflammatory responses. However, in active inflammatory bowel disease there is an increase in the mucosal macrophage population derived from circulating monocytes. These recruited macrophages differ phenotypically from the normal resident population of macrophages and play a major role in mediating the chronic mucosal inflammation seen in subjects with ulcerative colitis and Crohn's disease. The release of reactive metabolites of oxygen as well as nitrogen and proteases by macrophages may contribute to tissue injury (Mahida, Inflamm. Bowel Dis., February;6 (1):21-33 (2000)). Inflammatory mediators and more specifically reactive oxygen species have been shown to play an important pathogenic role in injury to the central nervous system and in arthritis and an increased production of oxygen free radicals has been observed in blood monocytes and alveolar macrophages from asthmatic subjects (Chanez et al., Am. Rev. Respir. Dis., 146: 1161-1166 (1992); and Vachier et al., J. Biolumin. Chemilumin., 9: 171-175 (1994)). Septic shock is another condition associated with stimulated mononuclear phagocytes.

Septic shock results from uncontrolled, sequential release of mediators having proinflammatory activity from cells following infection with gram negative or gram positive bacteria, and in response to endotoxins. See, e.g.; Tracey et al., Science, 234:470 (1986); Alexander et al., J. Exp. Med., 173:1029 (1991); Doherty et al., J. Immunol., 149:1666 (1992); Wysocka et al., Eur. J. Immunol., 25:672 (1995). Endotoxins exert their effects by inducing potent macrophage stimulation, and release of cytokines such as TNF-α, IL-1β, IL-6, IL-12, and IFN-γ. See Van Deuren et al., J. Pathol., 168:349 (1992). In particular IL-12, in concert with TNF-α, or B7 co-stimulation, can act as a potent inducer of IFN-γ production by T and NK cells. See D'Andrea et al., J. Exp. Med., 178:1041 (1993); Murphy et al., J. Exp. Med., 180:223 (1994), and; Kubin et al, J. Exp. Med., 180:211 (1994). The central role of proinflammatory cytokines in the pathogenesis of endotoxic shock is underlined by the occurrence of high levels of circulating cytokines in both humans and experimental animals during endotoxemia. See Stevens et al., Curr. Opin. Infect. Dis., 6:374 (1993). Cytokine triggering of regulatory mechanisms during sepsis may oppose macrophage stimulation (Heumann et al., Curr. Opin. Infect. Dis., 11 :279 (1998)). Both interleukin-10 (“IL-10”), and interleukin-4 (“IL-4”) have been shown to be efficacious in treatment of septic shock and LPS induced pathology. See, e.g., Marchant et al., Eur. J. Immunol., 24:1167 (1994); Howard et al., J. Exp. Med., 177:1205 (1993); Gerard et al., J. Exp. Med., 177:547 (1993); Baumhofer et al., Eur. J. Immunol. 28:610 (1998), Jain-Vora et al., Infect. Immun. 66:4229 (1998), and Giampetri et al., Cytokine 12: (2000). U.S. patent application Ser. No. 09/490,825 (incorporated herein by reference) filed Jan. 25, 2000, discloses the induction of IL-10 by IL-9 and proposes a role for IL-9 in the treatment of septic shock and endotoxemia.

A substantial body of literature demonstrates that anti-cytokine action can improve the outcome of subjects challenged by LPS or gram negative bacteria. See for example Beutler et al., Science, 229:689 (1985) and Heinzel et al., J. Immunol., 145:2920 (1990) who disclose the effects associated with the administration of neutralizing anti-cytokine antibodies, Ohlsson et al., Nature, 348:550 (1990) who disclose the effects of administering IL-IR antagonists, Bozza et al., J. Exp. Med., 189:341 (1999) who teach targeting of genes encoding proinflammatory cytokines, and both Pfeffer et al., Cell, 73:457 (1993), and Car et al., J. Exp. Med., 79:1437 (1994), who teach that administration of cytokine receptors can diminish lethality in experimental endotoxemia.

It is desireable to develop a method that would inhibit or prevent the stimulation of mononuclear phagocytes in response to factors which lead to oxidative burst and cytokine release, which in turn lead to, e.g., tissue damage, sepsis, an overwhelming, dysregulated inflammatory response, and the potential death of the subject. The results presented herein for the first time identify mononuclear phagocytes as new targets for IL-9.

### SUMMARY OF THE INVENTION

Described herein is the effect of IL-9 on the stimulation of mononuclear phagocytes, e.g., peripheral blood monocytes (PBM) and alveolar macrophages (AM), in response to an agent that activates mononuclear phagocytes resulting in the production of ROI and alterations in the production and/or release of various cytokines. IL-9 is shown herein to inhibit oxidative burst and to lead to altered levels of cytokine release and/or production, e.g., IL-9 promotes release of TGF-β and inhibits release of TNF-α, from stimulated mononuclear phagocytes. Stimulation of mononuclear phagocytes has been associated with various pathologic conditions, e.g., allergic inflammatory disorders of the bowel, various forms of eczema, autoimmune diseases, articular tissue damage, damage to lung tissue, liver tissue or tissue of the central nervous system, atherosclerotic plaque formation and septic shock. Many animal models for disorders associated with macrophage stimulation are available and routinely used in the art (see e.g. Mulligan supra for lung injury; Gross et al., Hepalogastroenterology, 41: 320-327 (1994) for inflammatory intestinal injury; Green et al., J. Cereb Blood Flow Metab., 21: 374-384 (2001), and Chan J. Cereb. Blood Flow Metab., 21:2-14 (2001) for central nervous system, and; Kawai et al., J. Dent. Res., 79: 1489-1495 for arthritis (2000)).

Thus the methods of this invention relate to preventing or inhibiting mononuclear phagocyte activation and thus preventing or inhibiting oxygen radical production and altering the levels of cytokines produced by stimulated mononuclear phagocytes in vitro and in vivo. The methods comprise treating the cells with IL-9 for a sufficient time to inhibit mononuclear phagocyte stimulation and thus inhibit oxygen radical production and alter the level of cytokine production and release by the stimulated mononuclear phagocytes. The results presented herein demonstrate that IL-9 treatment inhibits stimulation of mononuclear phagocytes and thus is useful for treating of a variety of pathologic disorders associated with activated mononuclear phagocytes. As such, an embodiment of this invention is a method for treating a subject having a pathologic disorder, or at risk of developing a pathologic disorder, that is associated with stimulated mononuclear phagocytes, comprising administering an effective amount of IL-9, or a portion of IL-9 or IL-9 derivative that can bind to IL-9 receptors, to a subject having such pathologic condition, or at risk of developing the pathologic condition, for a sufficient time to inhibit the onset and/or progression of the pathologic disorder. Preferably the subject is a mammal and preferably the mammal is a human.

### BRIEF DESCRIPTION OF THE FIGURES

FIG. 1 depicts the effect of IL-9, IL-4 and IFN-γ on O₂⁻ release by PMA-stimulated PBM. Data are means±SEM obtained from 3 experiments, triplicate conditions being performed in each experiment. *P<0.05 compared with cells preincubated with medium for the same period of time before the stimulation by PMA.

FIGS. 2A and B depict the effect of IL-9, IL-4, and IFN-γ on the intracellular oxidative burst in LPS-stimulated PBM (A) and AM (B). Data are means±SEM obtained from five (PBM) and three (AM) experiments, triplicate conditions being performed in each experiment. *P<0.001 compared with unstimulated cells; **P<0.001 compared with cells preincubated with medium alone before the stimulation by LPS

FIGS. 3A and B depict the effect of IFN-γ on the inhibition mediated by IL-9 and IL-4 on the oxidative burst in LPS-stimulated PBM (A) and AM (B). Data are means±SEM obtained from three experiments (n=3), duplicate conditions being performed in each experiment. *P<0.001 compared with cells preincubated with IL-4 alone.

FIGS. 4A-D depict a FACS analysis of IL-9R expression (A and C) and IL-9 binding (B and D) by PBM and AM. Insets in A and D depict confocal microscopy of PBM (A) and AM (D) stained for IL-9R.

FIGS. 5A-D depict the effect of IL-9, IL-4, and IFN-γ on the release of TNF-α (A and C) and IL-8 (B and D) by LPS-stimulated PBM and AM. Data are means±SEM (n=3). *P<0.001 compared with unstimulated cells; **P<0.05 compared with cells preincubated with medium.

FIGS. 6A-D depicts the effect of IL-9, IL-4, and IFN-γ on the release of IL-10 (A and C) and TGF-β1 (B and D) by LPS-stimulated PBM and AM cultured in the same conditions as described in FIG. 5. Data are means±SEM (n=3). *P<0.05 compared with unstimulated cells; **P<0.05 compared with cells preincubated with medium.

FIG. 7 depicts LPS-induced ERK phosphorylation (A) and the effect of PD98059 on LPS-stimulated oxidative burst (B) in PBM. Data are means±SD (n=3), and are representative of two experiments. *P<0.001 compared with cells treated with DMSO alone, without PD98059.

FIG. 8 depicts the effect of IL-9, IL-4 and TGF-β1 on ERK phosphorylation in PBM.

FIG. 9 depicts the effect of anti-TGF-β1 mAb and protein phosphatase inhibitors on cytokine-mediated ERK inhibition in PBM.

### DETAILED DESCRIPTION OF THE INVENTION

The results presented herein identify human monocytes/macrophage as a target for IL-9. IL-9 has been shown to affect mast cells, T and B lymphocytes, hematopoietic progenitors and lung epithelial cells, but no effect of this Th2 cytokine on mononuclear phagocytes has been reported to date. As disclosed herein, IL-9 exhibits inhibitory properties on several important monocyte/macrophage functions such as respiratory burst and cytokine release. For example, IL-9 inhibits the production of reactive oxygen intermediates (ROI), such as H₂O₂ and O₂⁻, by activated human blood monocytes and alveolar macrophages after, respectively, LPS and PMA stimulation. As disclosed herein, IL-9 pre-treatment of mononuclear phagocytes inhibits oxidative burst, even in the presence of IFN-γ, and alters the levels of cytokine release, e.g., TNF-α, IL-10 and TGF-β1, as compared to mononuclear phagocytes that were not pretreated with IL-9. This result is similar to IL-4 pretreatment effects on mononuclear phagocytes (Abramson and Gallin, J. Immunol., 144:625-630 (1990)). Preferably the stimulation of the mononuclear phagocytes is inhibited by incubating the cells with IL-9 prior to their stimulation with, e.g., LPS or PMA. More preferably, IL-9 is administered prophylactically, for example, for 24 hours before stimulation with a stimulatory agent, e.g., LPS or PMA.

Inhibition of the release of inflammatory mediators including TNF-α by LPS stimulated monocytes has been described for other Th2 cytokines, e.g., IL-10 and IL-13 (de Waal Malefyt et al., J. Exp. Med., 174:1209-1220 (1991) and de Waal Malefyt et al., J. Immunol., 151:6370-6381 (1993)). The inhibitory effect of IL-9 reported herein was specifically abolished by a blocking anti-hIL-9R mAb (the presence of IL-9 receptors was demonstrated on human mononuclear phagocytes by FACS) indicating that the response was due to the specific interaction of IL-9 with its receptor. Also disclosed herein is the presence of specific receptors for IL-9 on human mononuclear phagocytes. The presence of the receptors were detected using anti-hIL-9Rα mAbs and chimeric IL-9 protein.

This invention relates to methods for treating a subject who has, or is at risk for developing, a pathologic disorder associated with stimulated mononuclear phagocytes, for example, inflammatory disorders of the bowel, various forms of eczema, autoimmune diseases, articular tissue damage, damage to lung tissue, liver tissue or tissue of the central nervous system, atherosclerotic plaque formation and septic shock. Those of skill in this art appreciate that many pathologic disorders are induced, or the symptoms aggravated, by certain environmental triggers and in some cases a genetic predisposition for the development of such disorders is known to exist. For example, allergy symptoms are induced in response to particular antigens, e.g., animal dangers, pollen, dust mites and certain foods. The onset and progression of other conditions such as e.g., ulcers, inflammatory bowel diseases and damage to liver tissue, are associated with the consumption of certain foods or toxins, e.g. alcohol, carbon tetrachloride or caffeine. Still other disorders such as atherosclerosis are thought to be initiated by physical damage to arterial tissue, which leads to mononuclear phagocyte recruitment and stimulation and the development of atherosclerotic plaques. Thus one who is familiar with the triggers for these various pathologic disorders can determine if a subject is at risk for developing such disorders.

The effects of IL-9 on mononuclear phagocyte stimulation, e.g., an inhibition of oxidative burst, altered levels of released cytokines and inhibition of Extracellular signal-Regulated Kinase Mitogen-Activated Protein Kinase (ERK MAPK) activation, indicate that IL-9 pretreatment can inhibit or prevent tissue injury in subjects by stimulated mononuclear phagocytes. In particular the results presented herein demonstrate the benefits of IL-9 treatment in preventing tissue injury by e.g., oxidants and other proinflammatory mediators, e.g. TNF-α. Thus this invention also relates to methods of treating a pathological disorder associated with tissue injury by oxygen radicals and proinflammatory mediators, including when these are produced by stimulated mononuclear phagocytes. The methods comprise treating a subject having the pathological disorder, particularly the early stages of the disorder, or at risk of developing the disorder, with IL-9, or a portion of IL-9 or an IL-9 derivative that can bind to IL-9 receptor, wherein the IL-9 is administered for a time sufficient to reduce the stimulation of mononuclear phagocytes and inhibit their production of oxygen radicals. Preferably, the subjects are treated with IL-9 prior to exposure to an agent that would stimulate mononuclear phagocytes and inhibit or prevent the onset or progression of tissue injury in the treated subjects. The methods of this invention are suitable for treating a pathological condition associated with injury to a target tissue, e.g., liver tissue, lung tissue, vascular tissue, mucosal tissue, e.g. intestinal tissue, tissue of the central nervous system, joint and muscle tissue and cardiovascular tissue, wherein the injury to associated with oxygen radical and proinflammatory mediators produced by stimulated mononuclear phagocytes.

Oxygen radicals also play a role in active episodes of small-intestinal ischemia, ulcerative colitis, pancreatitis and gastric ulcer (Otamiri and Sjodahl, Dig. Dis., 9 (3) 133-41 (1991)). Thus the methods of this invention are useful for treating, e.g., small-intestinal ischemia, ulcerative colitis, pancreatitis and gastric ulcer. These methods comprise administering an effective amount of IL-9, or a portion of IL-9 or an IL-9 derivative that can bind to IL-9 receptors, and for a sufficient time to the subject at risk for developing the pathologic disorder or having the pathologic disorder, particularly a subject in the early stages of the disorder, wherein the IL-9 administration is sufficient to inhibit or prevent the stimulation of monocytes/macrophages thereby reducing the release of oxygen radicals and inhibiting the onset and progression of the disorder. Such treatment would prevent or alleviate the symptoms of the disorder, particularly small-intestinal ischemia, ulcerative colitis, pancreatitis and gastric ulcer.

The production of oxygen radicals by macrophages in response to LPS is increased in subjects with inflammatory bowel disease. There is a large population of macrophages in the normal intestinal mucosa and studies indicate that the normal intestinal macrophages cannot be easily induced to mediate acute inflammatory responses. However, in active inflammatory bowel disease there is an increase in the mucosal macrophage population, derived from circulating monocytes. These recruited macrophages differ phenotypically from the normal resident population of cells and play a major role in mediating the chronic mucosal inflammation seen in subjects with ulcerative colitis and Crohn's disease. The release of reactive metabolites of oxygen as well as nitrogen and proteases by macrophages may contribute to tissue injury. See Mahida, Inflamm. Bowel Dis., February;6 (1):21-33 (2000).

The results presented herein indicate that subjects having inflammatory bowel disease, particularly the early stages of this disorder, or at risk of developing this disorder, would benefit from treatment with IL-9. Thus another embodiment of this invention is a method for treating a subject having, or at risk for developing, an inflammatory bowel disease. The method comprises administering an effective amount of IL-9, or a portion of IL-9 or an IL-9 derivative that can bind to IL-9 receptors, to the subjects for a sufficient time to prevent or inhibit stimulation of mononuclear phagocytes. The amount of IL-9 is sufficient to reduce the production of ROI or to alter the levels of released cytokines, particularly TNF-α, by the mononuclear phagocytes, particularly those of the mucosal macrophage population. The reduced level of monocyte stimulation, e.g., a reduction in oxygen radical production and altered levels of cytokine production, would reduce damage to intestinal tissue of such subjects and inhibits the onset and reduces the symptoms of the disease.

This invention further relates to methods for treating a subject having, or at risk of developing, endotoxemia and sepsis. For example, the methods are useful for a subject having an infection, particularly a viral or bacterial infection, or at risk of developing a viral or bacterial infection. The methods are also useful for treating a subject undergoing a medical procedure where the risk for exposure to an agent that would promote activation of mononuclear phagocytes, e.g. bacterial or viral infection, can result in deadly consequences, e.g., septic shock. Such medical procedures include for example, transfusions, transplantations, chemotherapy, radiation therapy, immunotherapy, immunizations with antigens that the subject may or may not have received previously, ischemia reperfusion, or perfusions or infusions of compositions containing a compound that triggers antibody Fc receptors, e.g., an antigen-antibody complex. The methods comprise administering an effective amount of IL-9, or a portion of IL-9 or an IL-9 derivative that can bind to IL-9 receptors, to a subject having sepsis or at risk of developing sepsis wherein the IL-9 is administered for a sufficient time to prevent or inhibit the onset or progression of sepsis. Preferably the subject is treated with IL-9 prior to contact with an agent that would stimulate mononuclear phagocytes. Preferably the IL-9 is administered to the subject at least 24 hours prior to exposure to an agent that stimulates mononuclear phagocytes, e.g., prior to undergoing a medical procedure. The IL-9 may be administered to the subject up to about 96 hours prior to exposure to an agent that stimulates mononuclear phagocytes, e.g., prior to undergoing a medical procedure. The IL-9 is administered at an effective amount, which is sufficient to prevent or inhibit stimulation of mononuclear phagocytes, e.g., the effective amount inhibits oxidative burst or cytokine release in peripheral blood monocytes or alveolar macrophages.

TNF-α production by PBM is associated with a variety of pathologic disorders, such as, e.g., injury to liver, lung, CNS and intestinal tissue. Thus another embodiment of this invention relates to methods for treating a subject at risk for tissue injury as a consequence of TNF-α release from PBM by inhibiting the production of TNF-α by stimulated PBM. The method comprises contacting the PBM with an effective amount of IL-9 to inhibit their stimulation. The PBM may be contacted with the IL-9 in vitro or in vivo. For example, a sample of isolated PBM or a sample containing PBM, e.g., blood or tissue, may be contacted in vitro with a sufficient amount of IL-9 and for sufficient duration to inhibit production of TNF-α by the stimulated cells. Alternatively, the IL-9 may be administered to a subject at risk for tissue injury as a consequence of TNF-α release from PBM wherein the IL-9 is administered in sufficient quantity and for sufficient duration to inhibit the production of TNF-α from the stimulated mononuclear phagocytes. Preferably the PBM are contacted with the IL-9 for at least 24 hours, more preferably about 24 to 96 hours prior to stimulation.

Inflammatory mediators, and more specifically reactive oxygen species, have also been shown to play an important pathogenic role in injury to the central nervous system and in arthritis. Thus, another embodiment of this invention is a method for treating a subject at risk for arthritis or injury to tissue of the central nervous system, or treating subjects having these disorders, particularly those in early phase of these disorders, by administering an effective amount of IL-9, or a portion of IL-9 or an IL-9 derivative that can bind to IL-9 receptors, for a sufficient time to the subject, wherein the IL-9 administration is sufficient to inhibit or prevent the stimulation of monocytes/macrophages and thus reduce the release of oxygen radicals and prevent or inhibit articular injury or injury to tissue of the central nervous system.

Interferon gamma (IFN-γ) primes mononuclear phagocytes such that their functions, e.g., oxidative burst, altered levels of cytokine release and altered expression levels of CD14, in response to a stimulatory agent are enhanced as compared to control mononuclear phagocytes, e.g., mononuclear phagocytes that are not primed with IFN-γ prior to contact with the stimulatory agent. The priming effect of IFN-γ on mononuclear phagocytes is inhibited by coincubating the cells with IL-9 prior to exposure to the stimulatory agent. Thus another embodiment of this invention is a method for antagonizing IFN-γ's priming effect on the functions of mononuclear phagocytes in a subject whose mononuclear phagocytes are or will be primed by IFN-γ. The method comprises contacting mononuclear cells primed with IFN-γ with IL-9 prior to contacting the mononuclear phagocytes with the stimulatory agent. Preferably the phagocytes are contacted with a sufficient amount of IL-9 for at least 24 hours, prior to contact with the stimulatory agent.

An increased production of oxygen free radicals has been observed in blood monocytes and alveolar macrophages from asthmatic subjects (Chanez et al., Am. Rev. Respir. Dis., 146: 1161-1166 (1992) and Vachier et al, J. Biolumin. Chemilumin., 9: 171-175(1994)). This suggests using IL-9 to prevent macrophage activation in subjects with asthma, but many studies demonstrate IL-9, as well as of other Th2 cytokines, have a deleterious effect on this airway disease, probably related to activities of these cytokines on target cells of allergic inflammation. In addition, alveolar macrophages from asthmatics—which are primed for the release of ROI and cytokines—are less prone to both LPS stimulation and IL-4 down regulation than those from controls (Chanez et al., J. Allergy Clin. Immunol., 94:997-1005(1994)). Without wishing to be bound by theory, IL-9 might oppose the stimulatory effects of Thl-related agents, such as LPS or IFN-γ, on monocytes/macrophage while potentiating effects of molecules such as allergens that drive a Th2 response.

In contrast to asthma, IL-9 would be useful for preventing ARDS (Acute Respiratory Distress Syndrome). In a mouse model of acute lung injury induced by immune complexes, ROI have been shown to mediate tissue damage and both IL-4 and IL-10 exert an beneficial effect on this disorder (Mulligan et al., J. Immunol 1993; 151: 5666-5674 (1993)). Thus, another embodiment of this invention is a method for preventing the stimulation of monocytes/macrophages in a subject with ARDS or at risk for ARDS by administering an effective amount of IL-9, or a portion of IL-9 or an IL-9 derivative that can bind to IL-9 receptors, for a sufficient time to the subjects. The IL-9 is administered for a sufficient time to inhibit mononuclear phagocyte stimulation and reduce the release of oxygen radicals and alleviate or prevent the symptoms of ARDS.

The methods of this invention also relate to inhibiting the activation of Extracellular signal-Regulated Kinase Mitogen-Activated Protein Kinase (ERK MAPK) in mononuclear phagocytes. LPS induces ERK½ phosphorylation in PBM. This phosphorylation is prevented by pretreating the cells with a specific inhibitor of ERK kinase, PD98059. IL-9 pretreatment of PBM prior to LPS treatment strongly down regulates the level of ERK½ phosphorylation demonstrating that IL-9 preincubation of mononuclear phagocytes leads to the inhibition of ERK MAPK activation. Thus an embodiment of this invention is a method to inhibit ERK MAPK activation in a subject having a pathologic disorder associated with mononuclear phagocyte activation and thus inhibit the activation of mononuclear phagocytes and production of ROI in the subject. The method comprises contacting a sample containing containing cells that express ERK MAPK and IL-9 receptors with IL-9 prior to contacting the cells with an agent that promotes ERK phosphorylation. In this embodiment, the mononuclear phagocytes are contacted with an effective amount of IL-9 and for a sufficient duration such that the phosphorylation of ERK is inhibited as compared to mononuclear phagocytes that have not been preincubated with IL-9. The mononuclear phagocytes may be pretreated for at least 24 hours, with an effective amount of IL-9 prior to contacting said sample of mononuclear phagocytes with an agent that promotes phosphorylation ERK. An agent that promotes ERK phosphorylation in the cells of a subject may be, a cytokine, e.g., IFN-γ, TNF-α; an organic agent e.g., components of bacteria and viruses, such as viral coats, bacterial cell walls, membranes, enzymes, endotoxins, lipopolyssacharides (LPS), lipoteichoic acid (LTA), FcR triggering agents, phagocytosed particles, e.g., opsonized zymosan; a chemical agent for example phorbol myristate acetate (PMA), alcohol, carbon tetrachloride; or a physical agent, e.g., a hemodialysis membrane or tubing.

In the methods of this invention, an effective amount IL-9 is administered to a subject in need thereof for a sufficient time to inhibit mononuclear phagocyte stimulation. Preferably the IL-9 is administered prior to contacting the mononuclear phagocytes with an agent the promotes stimulation of the phagocytes. Preferably the IL-9 is administered to subjects for at least 24 hours prior to stimulation of mononuclear phagocytes.

The IL-9 may be administered with any pharmaceutically acceptable carrier and in any pharmaceutically acceptable route known in the art. As used herein, “pharmaceutically acceptable carrier” refers to any carrier, solvent, diluent, vehicle, excipient, adjuvant, additive, preservative, and the like, including any combination thereof, that is routinely used in the art. The carrier may contain other pharmaceutically acceptable excipients for modifying or maintaining pH, osmolarity, viscosity, clarity, color, sterility, stability, rate of dissolution, and/or odor. Similarly, the carrier may contain still other pharmaceutically acceptable excipients for modifying or maintaining the stability, rate of dissolution, release, or absorption or penetration across the blood-brain barrier. Further, the IL-9 may be combined with one or more therapeutically effective material for treatment of the pathologic disorders wherein the symptoms of the disorder or damage to tissue is associated with activated mononuclear phagocytes.

In the methods of this invention, the IL-9 may be delivered to the subjects systemically or locally to a target tissue. The IL-9 may be administered continuously or intermittently by inhalation or injection, e.g., subcutaneously, intravenously, intramuscularly, intrasternally, intrathecally, and intracerebrally, or orally, sublingually or by using infusion or perfusion techniques. Preferably the IL-9 is administered until the symptoms of the pathologic disorder are alleviated.

IL-9 is a well-characterized interleukin and portions of IL-9 or derivatives of IL-9, e.g., polypeptide products encoded by the DNA sequences of IL-9 wherein the DNA sequences contain various mutations, e.g., point mutations, insertions, deletions, or spliced variants of IL-9, which bind to the IL-9 receptor are known to those of skill in the art. See for example U.S. Pat. No. 6,261,559 to Levitt et al. (Assignee Geneara Corporation). The IL-9 may be naturally occurring, or recombinant in source, and may or may not be glycosylated. Preferably the IL-9 is a human IL-9.

Those of skill in the art appreciate that there are a variety of agents that activate mononuclear phagocytes. For example, the agent may be an organic material, e.g., components of bacteria and viruses, such as viral coats, bacterial cell walls, membranes, enzymes, lipopolyssacharides (LPS), the agent may be a chemical agent such as for example phorbol myristate acetate (PMA), alcohol or carbon tetrachloride, or a physical agent, e.g., by contact with a hemodialysis membrane or tubing or by ischemia reperfusion. In one embodiment a sample containing mononuclear phagocytes, e.g., blood, tissue or organs, may be treated with IL-9 prior to dialysis, infusion or transplantation into a subject in need thereof Alternatively the subject in need thereof may be treated with IL-9 prior to receiving a transfusion or infusion or prior to transplantation of a tissue or organ to alter the levels of cytokine production and/or release, e.g., TGF-β and TNF-α and to inhibit oxidative burst by the subject's own mononuclear phagocytes.

The inhibition of monocyte/macrophage activation by IL-9 is similar to that previously described for IL-4 (Abramson and Gallin, J. Immunol., 144:625-630 (1990) and Bhaskaran et al., J. Leukoc. Biol., 52:218-223 (1992)). However, in contrast to IL-4, the inhibition mediated by IL-9 is not abolished by IFN-γ. Becker and Daniel, Cell. Immunol., 129:351-362 (1990) disclose that IL-4 inhibition is abolished by IFN-γ. IFN-γ is known to prime monocytes, notably for the production of ROI. The absence of an antagonistic effect on IL-9 by IFN-γ is not due to a down regulation by IL-9 of the expression of IFN-γR on monocytes/macrophages. This major difference between deactivation by IL-9 and by IL-4 suggests that IL-9 uses a different mechanism to mediate its effect on monocytes/macrophages, a possibility supported by the additive effects of IL-9 and IL-4 observed on the PMA-stimulated oxidative burst in monocytes.

Cytokine release is a second monocyte/macrophage function modulated by IL-9. The results presented herein demonstrate that IL-9 pretreatment of mononuclear phagocytes reduces production of TNF-cc in response to LPS. This is similar to the effect of other cytokines, e.g., IL-4, IL-10, IL-13 and TGF-β, which also inhibit production of inflammatory mediators such as TNF-α in response to LPS (Hart et al., Proc. Natl. Acad. Sci. USA 86:3803-3807 (1989); de Waal Malefyt et al., J. Exp. Med., 174:1209-1220 (1991) and de Waal Malefyt et al., J. Immunol., 151:6370-6381 (1993) and; Tsunawaki et al., Nature, 334:260-262 (1988)). In contrast, IFN-γ has been reported to potentiate TNF-α release by LPS-activated monocytes (Joyce and Steer, Cytokine. 8:49-57 (1996)). While IL-9 inhibits the production of TNF-α it does not inhibit release of IL-8 by monocytes stimulated by LPS. This is in contrast to the effects of IL-4, which inhibits LPS-induced IL-8 release by monocytes (Wang et al., Blood. 83:2678-2683 (1994)) suggesting that IL-9 inhibits mononuclear phagocytes through a regulatory pathway distinct from that of IL-4.

To evaluate potential mechanisms for cytokine-mediated regulation of the oxidative burst and cytokine release the modulation of surface LPS receptors, CD 14 and TLR4, was analyzed, as was the regulation of IL-10 in LPS-stimulated monocytes. IL-4 down regulates CD14 expression on blood monocytes and alveolar macrophages (Hasday et al., Am. J. Physiol., 272:L925-933 (1997), and also significantly inhibited expression of TLR4. In contrast, IL-9 does not modulate surface expression of CD14 or TLR4 on monocytes.

IL-10 is a major monocyte/macrophage suppressing factor (de Waal Malefyt et al., J. Exp. Med., 174:1209-1220 (1991)) that inhibits the production of inflammatory mediators such as TNF-α and ROI by monocytes. Thus the regulation of IL-10 release by IL-9 was evaluated. IL-9 preincubation of monocytes down regulates LPS-induced production of IL-10. Thus another aspect of this invention is a method for inhibiting the production of IL-10 by stimulated peripheral blood monocytes (PBM) comprising contacting said PBM with an effective amount of IL-9 for a sufficient duration prior to contacting the PBM with an agent that stimulates the PBM wherein said effective amount of IL-9 is an amount sufficient to inhibit IL-10 production by PBM as compared to PBM that were not contacted with IL-9. Moreover, neutralization of IL-10 activity by anti-human IL-10R blocking mAb failed to abrogate the IL-9 effect, or the IL-4 effect, on the respiratory burst in LPS-stimulated monocytes. In addition, both IL-4 and IFN-γ suppressed LPS-induced IL-10 release by monocytes, supporting previous studies (Bonder et al., Immunol., 96:529-536 (1999); Bach and Brashler, Int. Arch. Allergy Immunol., 107:90-92 (1995)). Thus, the results presented herein indicate that IL-9 and IL-4 inhibit mononuclear phagocyte activation through IL-10 independent mechanisms.

IL-9's inhibition of respiratory burst in LPS-activated monocytes was significantly inhibited by a mAb neutralizing TGF-β but not by an anti-IL-10R mAb. This is in contrast to the inhibitory effect of IL-4, which appeared to be independent of TGF-β (Abramson and Gallin, J. Immunol., 144:625-630 (1990)). Moreover, IL-9 (and not IL-4) strongly potentiated the production of TGF-β by LPS-stimulated monocytes and alveolar macrophages. The results disclosed herein demonstrate that TGF-β down regulates oxidative burst in LPS-stimulated monocytes and is induced by IL-9 in LPS-activated monocytes/macrophages. Thus the inhibitory effect of IL-9 on the production of ROI is mediated at least in part by TGF-β.

The ERK MAP kinase pathway plays a key role in the control of monocyte/macrophage activation by LPS, as assayed by TNF-α release (Trotta et al., J. Exp. Med., 184:1027-1035 (1996). While ERK may regulate the phosphorylation of p₄₇^{phox}, a subunit of NADPH oxidase (Dewas et al., J. Immunol., 165:5238-5244 (2000)), induction of the oxidative burst in neutrophils by LPS depends only partly on this MAPK pathway (Bonner et al., Inf. Immun., 69:3143-3149 (2001)). ERK activation is necessary for the stimulation of the oxidative burst in monocytes by LPS, since PD98059, a specific inhibitor of ERK phosphorylation, completely suppressed the LPS effect on ROI production. IL-9 pretreatment inhibits ERK activation in LPS-stimulated monocytes, as does IL-4 treatment of human monocytes and TGF-β treatment of murine macrophages (see respectively, Niro et al., Biochem. Biophys. Res. Commun., 250:200-205 (1998) and Rose et al., Biochem. Biophys. Res. Commun., 238:256-260 (1997)). Moreover, and again in contrast with IL-4, the mechanism of ERK inactivation by IL-9 appeared dependent on both TGF-β and on serine/threonine phosphatase activity. ERK inhibition by TGF-β in pancreatic carcinoma cells was abrogated by okadaic acid, which is a protein phosphatase inhibitor (Giehl et al., Oncogene, 19:4531-4541 (2000)). Okadaic acid-sensitive protein phosphatase 2A dephosphorylates and deactivates ERK in vitro (Anderson et al., Nature, 343:651-653 (1990)). Thus, in contrast with IL-4, which affects LPS binding to monocytes, the results presented herein indicate that IL-9 inhibits LPS-stimulation of human monocytes through a TGF-β-mediated dephosphorylation of ERK½ MAP kinases.

Other features of the invention will be clear to the artisan and need not be discussed further.

The terms and expressions and following examples which have been employed are used as terms of description and not of limitation, and there is no intention in the use of such terms, expressions and examples of excluding any equivalents of the features shown and described or portions thereof, it being recognized that various modifications are possible within the scope of the invention.

### EXAMPLES

I. Effect of IL-9 on Stimulated Mononuclear Phagocytes

A. IL-9 Inhibition of Oxidative Burst

Intracellular oxidative capacity was assessed essentially as described by Bass et al., J. Immunol., 130:1910-1917 (1983) (incorporated herein by reference) as described infra.

Extracellular release of O₂-derived radicals was evaluated by the SOD-inhibitable reduction of ferricytochrome c, as previously described (Pick, Methods in Enzymology, 132:407-421 (1986)) as described infra.

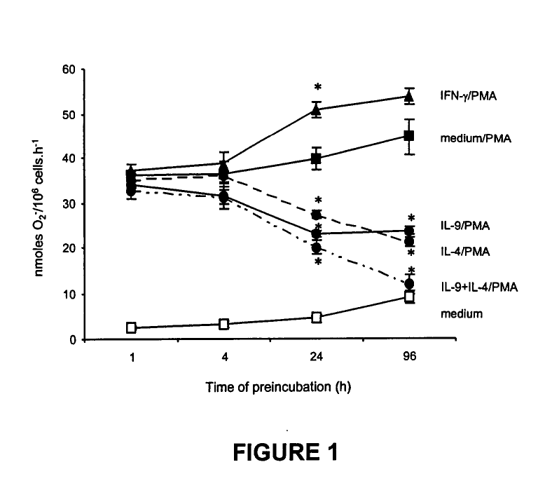

In preliminary experiments, PMA was shown to exert only a minor effect on DCFH oxidation but strongly stimulated cytochrome c reduction by PBM (FIG. 1). While incubation for 24 hour with IL-9 had little effect on the basal oxidative burst, as also observed with IL-4, preincubation for 24 hour with IL-9 significantly down regulated the O₂⁻ release by PMA-stimulated PBM (23.2±0.8 vs 40.1±2.5 nmoles 02-per 10⁶ cells. h⁻¹, p<0.001) (FIG. 1). The effect of IL-9 was not significant for shorter preincubation periods (1 hour, 4 hour) and was not further increased for 96 hour of preincubation (FIG. 1). The same inhibitory effect was observed with IL-4 which also started at 24 hour of preincubation but was significantly increased after 96 hour (FIG. 1). Moreover, an additive effect was observed between IL-9 and IL-4 after 96 hour of preincubation. In contrast to IL-9 and IL-4, IFN-γ slightly up regulated the O₂⁻ release by PMA-stimulated PBM after 24 hour of preincubation (FIG. 1).

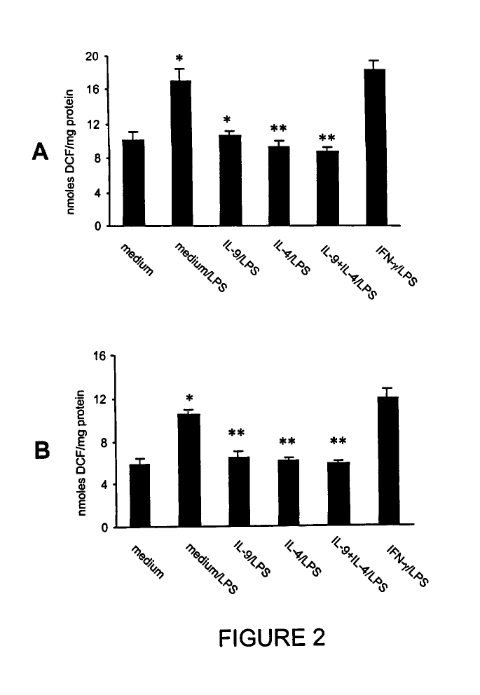

Stimulation by LPS for 20 hour increased DCFH oxidation (and cytochrome c reduction) about two-fold as compared with unstimulated cells, both in PBM and AM (FIG. 2). Preincubation for 24 hour with IL-9 down regulated the LPS-stimulated oxidative burst in PBM (10.7±0.5 vs 17.0±1.3 nmoles DCF/mg protein, p<0.001) and in AM (6.5±0.5 vs 10.4±0.3 nmoles DCF/mg protein, p<0.001) (FIG. 2) to its baseline level. A similar inhibitory effect was observed with IL-4 both in PBM and AM (FIG. 2). In contrast, preincubation with IFN-γ slightly increased the oxidative burst although this effect was not statistically significant in LPS-stimulated PBM and AM (FIG. 2).

B. Effect of IFN-γ on IL-9 Inhibition

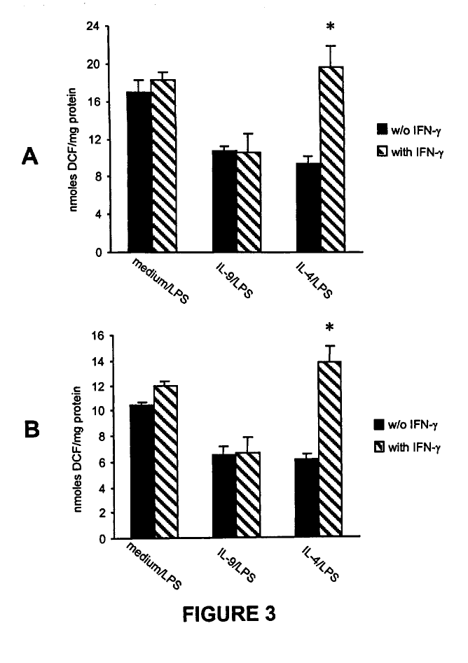

Cells (0.2×10⁶) were preincubated for 24 hour with IL-9 or IL-4 (20 ng/ml) in the presence of IFN-γ (200 U/ml) before stimulation for 20 hour with LPS (1 μg/ml). The intracellular oxidative capacity was measured through the DCFH oxidation assay as describe herein. FIG. 3 depicts the effect of IFN-γ on the inhibition mediated by IL-9 and IL-4 on the oxidative burst in LPS-stimulated PBM (A) and AM (B).

The influence of IFN-γ on the IL-9 mediated inhibition, as well as the IL-4 mediated inhibition of the oxidative burst in LPS-stimulated mononuclear phagocytes was evaluated by co-incubating cells with IL-9 or IL-4 and IFN-Y. Inhibition of the respiratory burst by IL-9 was maintained in the presence of IFN-γ in both LPS-stimulated PBM (10.5±2.1 vs 10.7±0.5 nmoles DCF/mg protein respectively with and without IFN-γ, NS) and AM (6.6±1.2 vs 6.5±0.6 nmoles DCF/mg protein, NS) (FIG. 3). In contrast, IFN-γ abrogated the inhibitory effect of IL-4 on the oxidative burst in LPS-stimulated PBM (19.6±2.2 vs 9.4±0.6 nmoles DCF/mg protein respectively with and without IFN-γ, p<0.001) and in AM (13.8±1.3 vs 6.1±0.4 nmoles DCF/mg protein, p<0.001) (FIG. 3).

C. Effect of Neutralizing Anti-IL-9R MAB on IL-9 Inhibited Oxidative Burst

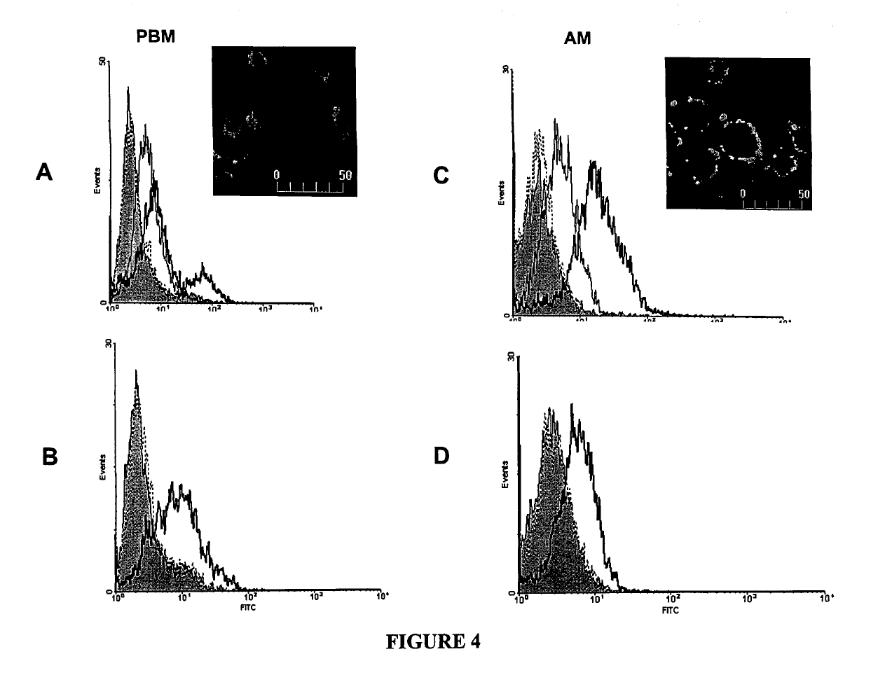

FIG. 4 depicts a FACS analysis of IL-9R expression (A) and IL-9 binding (B) by PBM and AM. PBM and AM cells (0.2×10⁶) were incubated with AH9R2 or AH9R7 mAb (10 μg/ml), and thereafter with SAM-FITC (10 μg/ml), as described infra. Autofluorescence and control histograms are represented in FIG. 4. IL-9R staining of PBM and AM with AH9R2 mAb was also evaluated by confocal microscopy (insets, scales are in micrometers), as described infra.

To assess the IL-9 binding by PBM and AM, cells (0.2×10⁶) were incubated with hIL-9-nIlgG3 chimeric protein and binding was assayed with GAM3-FITC. Autofluorescence and control histograms (control cells were incubated with mIgG3 followed by GAM3-FITC) are shown in FIG. 4. FIG. 4 is representative of five and three experiments for respectively IL-9R expression and IL-9 binding.

Preincubation of PBM and AM with neutralizing AH9R7 anti-hIL-9R mAb (10 μg/ml) 1 hour before IL-9 incubation abolished respectively 90% ±5 (means±SEM) and 87%±3 of the IL-9 effect on LPS-stimulated DCFH oxidation, in comparison with the absence of blockade by control mIgG2b (Table 1). Moreover, using the same mAb (as well as AH9R2 mAb), specific surface receptors for IL-9 were identified on PBM and AM by FACS (FIG. 4). A significant shift of the fluorescence histogram was observed when adherent PBM were incubated with AH9R7 mAb, as compared with cells incubated with control mIgG2b, and detected with SAM-FITC, as well as with AH9R2 mAb as compared with control mIgG2a (FIG. 4A). Similar results were observed for AM with AH9R2 and AH9R7 mAbs, as compared with their respective control mIgG. The same pattern of staining, more intense with AH9R2 than with AH9R7 mAb, was also obtained on hIL-9R-transfected Baf-3 cells. Expression of IL-9R by human mononuclear phagocytes was confirmed by confocal microscopic examination of PBM and AM stained by the same method with AH9R2 mAb (FIG. 4A and C, insets). In addition, a significant binding of IL-9 on the surface of PBM and AM was observed when these cells were incubated with chimeric IL-9-mIgG3 protein revealed by GAM3-FITC, as compared with control (FIG. 4B).

TABLE 1

| | Oxidative burst | |
|---|---|---|
| Cell treatment | (nmoles DCF/mg protein) | % of blockade |
| PBM | | |
| medium | 10.1 ± 0.4 | |
| LPS | 17.0 ± 0.3 | |
| IL-9/LPS | 10.7 ± 0.5 | |
| IL-9 + mIgG2b/LPS | 9.2 ± 0.4 | 0 |
| IL-9 + anti-IL-9R/LPS | 16.4 ± 0.3* | 90 ± 5 |
| AM | | |
| medium | 5.8 ± 0.6 | |
| LPS | 10.4 ± 0.3 | |
| IL-9/LPS | 6.5 ± 0.5 | |
| IL-9 + mIgG2b/LPS | 6.2 ± 0.4 | 0 |
| IL-9 + anti-IL-9R/LPS | 9.9 ± 0.3* | 87 ± 3 |

Table 1. Specific blockade of the IL-9 inhibitory effect on oxidative burst in LPS-15 stimulated PBM and AM by anti-hIL-9R mAb. Cells (0.2×10⁶) were preincubated with neutralizing mAb against hIL-9R (AH9R7, 10 μg/ml), or with control mIgG2b (10 μg/ml), 1 hour before incubation with IL-9 (20 ng/ml) for 24 hour without removing mAb, and stimulated by LPS (1 μg/ml) for 20 h. Intracellular oxidative capacity was evaluated by the DCFH oxidation assay. Data are means±SEM obtained from three experiments, triplicate conditions being performed in each experiment. *P<0.001 compared with cells preincubated with IL-9 (with or without control mIgG2b).

D. Effect of IL-9 on TNF-α and IL-8 Release by LPS-Stimulated PBM

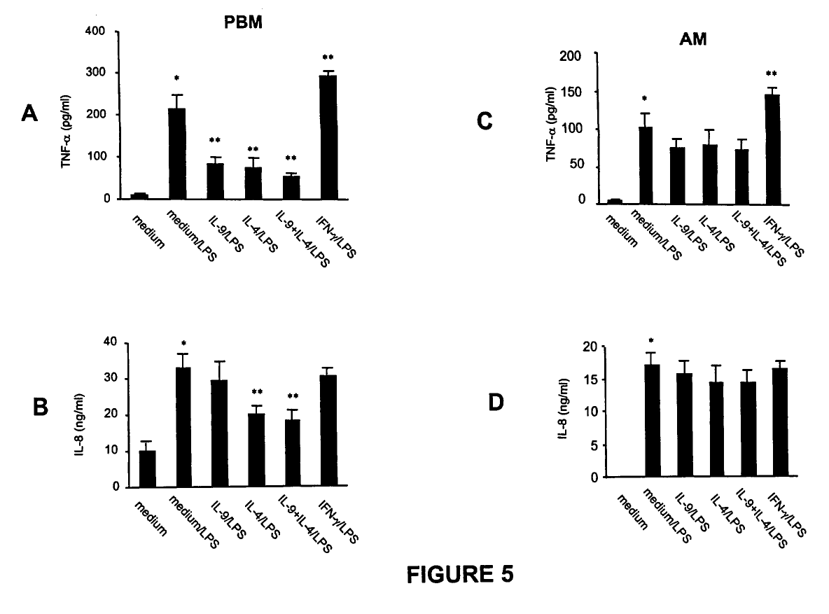

PBM and AM obtained as described infra (1×10⁶ PBM, 0.5×10⁶ AM) were preincubated for 24 hour with cytokines, IL-4, IL-9 (20 ng/ml) and IFN-γ (200 U/ml) before the stimulation by LPS (1 μg/ml) for 20 h. Supernatants were harvested and frozen at −20° C. until cytokine titration. TNF-α release was determined in supernatants by a cytotoxic bioassay using WEHI 164 clone 13 target cells (Espevik and Nissen-Meyer, J. Immunol. Methods, 95:99-105 (1986) (incorporated herein by reference)), while IL-8 was quantitated in the same supernatants by ELISA. FIG. 5 depicts the effects of IL-9, IL-4, and IFN-γ on the release of TNFα (A) and IL-8 (B) by LPS-stimulated PBM and AM. Data are means±SEM (n=3). *P<0.001 compared with unstimulated cells; **P<0.05 compared with cells preincubated with medium.

The release of TNF-α, which was constitutively very low especially in AM, was strongly increased by LPS both in PBM (212.4±34.1 vs 10.2±4.1 pg/ml respectively with and without LPS, p<0.001) and AM (102.4±17.6 vs 4.6±2.1 pg/ml, p<0.001) (FIG. 5A). PBM preincubated for 24 hour with IL-9 before the LPS stimulation released much less TNF-α than PBM preincubated with medium alone (84.2±17.2 vs 212.4±34.1 pg/ml, p=0.004). A similar effect was observed for PBM preincubated with IL-4 (72.5±21.5 vs 212.4±34.1 pg/ml, p=0.003), and the combination of IL-9 and IL-4 did not induce a significant increase of the inhibitory effect observed with each cytokine alone. No significant inhibition of TNF-α production occurred in LPS-stimulated AM preincubated with IL-9 (74.8±10.2 vs 102.4±17.6 pg/ml, NS), nor with IL-4 (FIG. 5A). In contrast with IL-9 and IL-4, IFN-γ potentiated the TNF-α release by LPS-stimulated PBM (289.8±12.9 vs 212.4±34.1 pg/ml respectively with and without IFN-γ, p=0.04) and AM (145.2+9.4 vs 102.4±17.6 pg/ml, p=0.04) (FIG. 5A). A constitutive release of IL-8 was observed in PBM but not in AM (FIG. 5B). As for TNF-α, incubation for 20 hour with LPS markedly up regulated the IL-8 release by PBM (32.8±4.4 vs 10.0±2.4 ng/ml respectively with and without LPS, p<0.001) and AM (16.9±0.8 vs<10 pg/ml, p<0.001). In contrast with IL-4 which inhibited the LPS stimulation of IL-8 production by PBM (20.1±2.1 vs 32.8±4.4 ng/ml respectively with and without IL-4, p=0.02) but not by AM, no significant modulation of the IL-8 release by PBM and AM was observed with IL-9, nor with IFN-γ (FIG. 5B).

E. IL-9 Does not Modulate Surface Expression of LPS Receptors

CD14 and TLR4 expression on the surface of PBM preincubated for 24 hour with IL-9 was not significantly different from their expression on PBM preincubated with medium alone (Table 2). In contrast, expression of both CD14 and TLR4 on PBM was down regulated by preincubation with IL-4, and increased by IFN-γ (Table 2). The up regulation of CD14 on PBM incubated with IFN-γ was inhibited by IL-9, but not by IL-4.

TABLE 2

| Cell treatment | CD14 expression (MFI) | TLR4 expression (MFI) |
|---|---|---|
| medium | 107 ± 4 | 73 ± 2 |
| IL-9 | 108 ± 5 | 71 ± 4 |
| IL-4 | 57 ± 3* | 58 ± 1* |
| IFN-γ | 121 ± 6* | 82 ± 2* |

Table 2. Cells (0.2×10⁶) were preincubated for 24 hour with cytokines (20 ng/ml for IL-9 or IL-4 and 200 U/ml for IFN-γ), before the assessment of LPS receptor expression by immunostaining using anti-human CD14 mAb conjugated to FITC and anti-human TLR4 rabbit Ab followed by mouse anti-rabbit IgG (MAR-FITC). Cell-associated fluorescence was evaluated by FACS and expressed as mean fluorescence intensity (MFI). Autofluorescence of PBM was negligible (MFI: 3±1). Data are means±SD (n=3), and are representative of two experiments. *P<0.05 compared with cells preincubated with medium.

F. Role of TGF-β1 in IL-9 Inhibition of Oxidative Burst

Cells (0.2×10⁶) were preincubated with neutralizing mAb, either against TGF-β1 (30 μg/ml) or against IL-10Rβ (30 μg/ml) or with control mIgG1 (30 μg/ml), 2 hour before incubation with IL-9 (20 ng/ml) for 24 hour without removing the mAb, and stimulated with LPS (1 μg/ml) for 20 h. Intracellular oxidative capacity was evaluated by the DCFH oxidation assay.

In order to assess the mechanism by which IL-9 inhibits oxidative burst in LPS-stimulated PBM, we determined if two known monocyte/macrophage deactivating factors, i.e., IL-10 and TGF-β1, were necessary for the inhibition. While anti-IL-10R neutralizing mAb failed to suppress the inhibitory effect of IL-9, preincubation of PBM with anti-TGF-β1 neutralizing mAb inhibited 56% ±5 of the IL-9 effect on the LPS-stimulated respiratory burst (14.2±0.4 vs 10.7±0.5 nmoles DCF/mg protein, p<0.001) (Table 3). Moreover, TGF-β1 was shown to inhibit the respiratory burst in LPS-stimulated PBM to the same extent as IL-9 (10.2±0.5 vs 16.2±0.4, p<0.001). In contrast to IL-9, the inhibition mediated by IL-4 was not significantly suppressed by anti-TGF-β1 mAb, nor by anti-IL-10R.

TABLE 3

| | Oxidative burst | |
|---|---|---|
| Cell treatment | (nmoles DCF/mg protein) | % of blockade |
| Medium | 10.3 ± 0.2 | |
| LPS | 16.2 ± 0.4 | |
| IL-9/LPS | 10.8 ± 0.6 | |
| TGF-β1/LPS | 10.2 ± 0.5 | |
| IL-9 + mIgG1/LPS | 10.5 ± 0.3 | 0 |
| IL-9 + anti-IL-10R/LPS | 11.0 ± 0.3 | 4 ± 2 |
| IL-9 + anti-TGF-β1/LPS | 14.2 ± 0.4* | 63 ± 5 |

Table 3. Blockade of the IL-9 inhibitory effect on oxidative burst in LPS-stimulated PBM by anti-TGF-β1 neutralizing mAb, and inhibitory effect of TGF-β1. Data are means±SD (n=3), and are representative of two experiments. *P<0.001 compared with cells preincubated with IL-9 without neutralizing anti-TGF-β1 mAb.

G. Effects of IL-9 on IL-10 and TGF-β1 Release

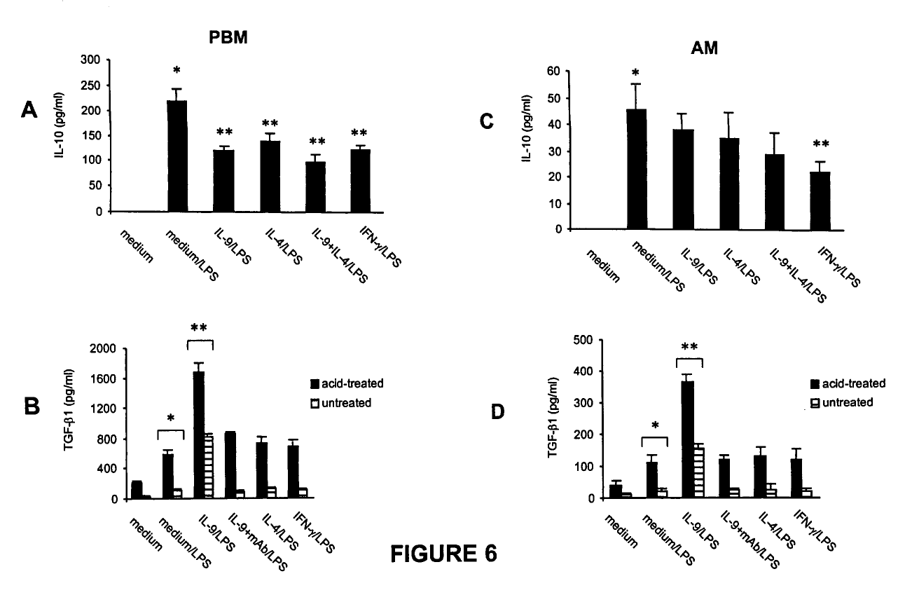

FIG. 6 depicts the effect of IL-9, IL-4 and IFN-γ on the release of IL-10 (A) and TGF-β1 (B) by LPS-stimulated PBM and AM cultured under the same conditions as described in FIG. 5 Example I. D. TGF-β1 was determined by ELISA in crude supernatants (untreated supernatants) and in ethanol-acid (acid-treated) supernatants, using a kit from Biosource for determining TGF-β1 release from its latent complexes. An ELISA kit from CLB (Amsterdam, The Netherlands) was used to determine IL-10 following the manufacturer's protocol. The effect of IL-9 on TGF-β1 production was specifically blocked by preincubating cells 1 hour before IL-9 treatment with anti-IL9R AH9R7 mAb (10 μg/ml). Data are means±SEM (n=3). *P<0.05 compared with unstimulated cells; **P<0.05 compared with cells preincubated with medium.

IL-10 was not detectable in supernatants from unstimulated mononuclear phagocytes, but IL-10 release was strongly induced by LPS both in PBM and AM (FIG. 6A). IL-9 down regulated the LPS-induced IL-10 release by PBM (119.7±8.7 vs 217.8±25.8 pg/ml respectively p=0.01), but not by AM (FIG. 6A). IL-4 also inhibited the IL-10 release by LPS-stimulated PBM but not by AM, whereas IFN-γ decreased the IL-10 release in both LPS-stimulated PBM and AM (FIG. 6A).

While no modulation of TGF-β1 was observed in unstimulated PBM treated with IL-9, the production of TGF-β1 by LPS-stimulated PBM was strongly potentiated by IL-9 (1687±94 vs 586±64 pg/ml in acid-treated supernatants, p<0.001) (FIG. 6B). This effect, not observed with IL-4 nor with IFN-γ, also occurred significantly in LPS-stimulated AM preincubated with IL-9 (366+21 vs 109±23 pg/ml in acid-treated supernatants, p<0.001) (FIG. 6B). Moreover, the IL-9-mediated TGF-β1 up regulation was specifically inhibited by the neutralizing anti-IL-9R mAb, but not with control mIgG2b both in PBM (857±89 vs 1687±94 pg/ml, p<0.001) and AM (118±14 vs 366±21 pg/ml, p<0.001) (FIG. 6B).

H. IL-9 Pre-Treatment Effects of ERK Phosphorylation

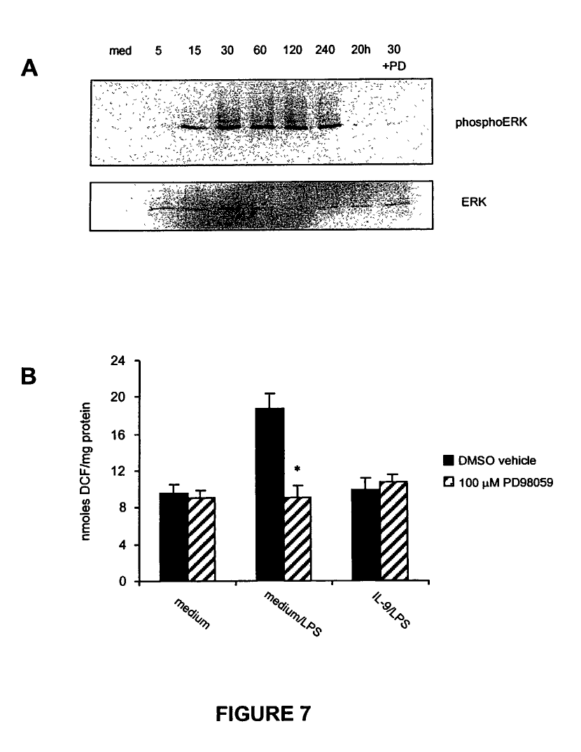

PBM, isolated as described infra, were treated with 100 !M PD98059 (or with the same volume of DMSO as control) 1 hour before preincubation for 24 hour with medium or IL-9 (20 ng/ml). For the phosphoERK immunoblot assay (A), PBM were then stimulated by LPS (1 μg/ml) for the indicated periods of time (5 min to 20 hours) as compared to unstimulated cells (med), and processed for the detection of phosphorylated and total ERK½ as described in infra. For the oxidative burst (B), PBM were stimulated by LPS (1 μg/ml) for 20 hour and their oxidative capacity was evaluated through DCFH oxidation The results are presented in FIG. 7.

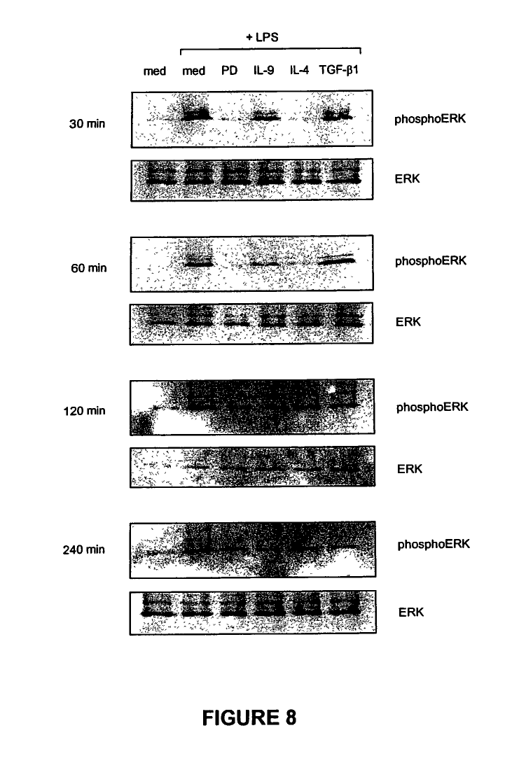

PBM were preincubated for 24 hour with medium alone (med) or IL-9, IL-4 or TGF-β1 (20 ng/ml), and stimulated by LPS (1 μg/ml) for the indicated periods of time (30 to 240 min). When indicated, PBM were pretreated with 100 μM PD980591 hour before LPS stimulation. Cell lysates were processed for the detection of phosphorylated and total ERK½ as described in infra. The results are presented in FIG. 8.

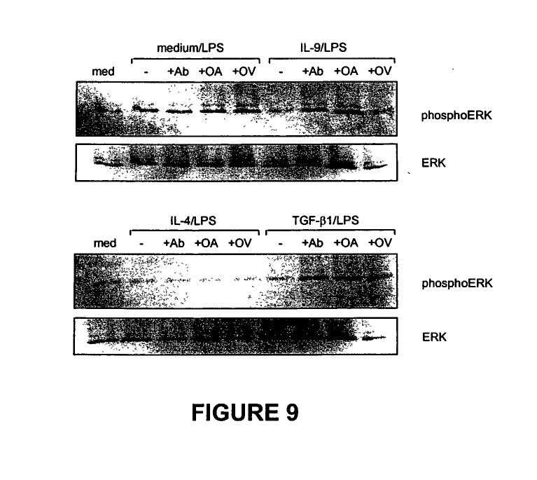

PBM were pretreated with 30 μg/ml anti-TGF-β1 mAb (Ab) for 2 hour (without being removed), or with 1 μM okadaic acid (OA) or 2.5 mM orthovanadate (OV) for 15 min (and removed), before preincubation with medium alone, IL-9, IL-4 or TGF-β1 (20 ng/ml) for 24 h. PBM were then stimulated by 1 μg/ml LPS for 240 min and lysed to assess phosphorylated and total ERK as described infra. The results are presented in FIG. 9.

II. Cells and Assays

A. PBM and AM Isolation

Human AM and PBM were obtained as follows: human AM were obtained from non-smoking healthy volunteers by bronchoalveolar lavage (BAL) by standard methods (Sibille et al., Am. Rev. Respir. Dis., 139:740-747 (1989)), incorporated herein by reference). All volunteers gave written consent and the BAL procedure was approved by the local ethical committee. Macrophages, which accounted for more than 90% of BAL mononuclear cells as determined by Giemsa-stained cells from cytospins, were incubated in plastic plates in complete RPMI (cRPMI, RPMI-1640 culture medium supplemented with 2 mM L-glutamine, 100 IU/ml penicillin, 100 μg/ml streptomycin, and 10% v/v heat-inactivated FBS) for 1 hour at 37° C. Nonadherent cells (mainly lymphocytes) were removed by washings with cRPMI.

PBM were obtained from whole blood of healthy blood donors and purified in a one-step density gradient method using Polymorphprep (Nycomed, Oslo, Norway) following the manufacturer's protocol. Blood mononuclear cells were incubated for 1 hour at 37° C. in cRPMI on plastic plates and non-adherent cells and lymphocytes were removed by three washings with cRPMI.

Mononuclear phagocytes (AM and PBM) represented more than 95% of total adherent cells upon microscopic examination of cytospins and flow cytometry of the purified cells. Cell viability assayed by trypan blue exclusion was at least 90% for the different experimental conditions.

B. Oxidative Burst Assay

Intracellular oxidative capacity was assessed as described by Bass et al., (Bass et al., J. Immunol. 130:1910-1917 (1983)). Briefly, the PBM and AM samples were distributed in 96-well plates with flat bottoms (Falcon) to provide 0.2×10⁶cells/well. The cells were preincubated for 24 hour at 37° C., 5% CO₂ with cytokines (200 u/ml for IFN-y, 20 ng/ml for IL-9 and 20 ng/ml; for IL-4) in cRPMI before being stimulated for 20 hours with LPS (1 μg/ml) without removing the cytokines. At the end of incubation with cytokines and/or LPS, cells were loaded for 15 min with 15 μM DCFH-DA in cRPMI which, after passive penetration into cells, is hydrolyzed into nonfluorescent polar DCFH and trapped inside the cells. DCFH is then oxidized into highly fluorescent DCF according to the intracellular amount of hydrogen peroxide (H₂O₂) produced by the respiratory burst. After three washings with phosphate-buffered saline (PBS, pH 7.4), cells were lysed in 0.1% v/v Triton X100 (Sigma) in PBS, and fluorescence was quantified in a computerized microplate spectrofluorimeter (Packard Instruments, Downers Grove, Ill., USA) at 485 nm excitation/530 nm emission wavelengths. DCF concentrations were deduced from a standard curve of known concentrations of fluorescent DCF (ranging from 0.08 to 10 μM in lysis buffer) Results were corrected for total protein concentration determined in cell lysates by the bicinchoninic acid-based method (Pierce, Rockford, Ill., USA), and were expressed as nmoles DCF/mg cell protein.

Extracellular release of O₂-derived radicals was evaluated by the SOD-inhibitable reduction of ferricytochrome c, as previously described (Pick, Methods in Enzymology, 132:407-421 (1986)). Briefly, after incubation with IL-9 and IL-4 (20 ng/ml) for 1 hour, 4 hour 24 hour and 96 hour, cells were washed three times in Hank's balanced salt solution without phenol red (HBSS) to remove the phenol red-containing medium and incubated at 37° C. with HBSS containing 160 μM ferricytochrome c (plus 300 IU/ml SOD as control for each condition), and concomittantly with 100 ng/ml PMA when indicated. Optical density (OD) at 550 nm was then recorded in a plate spectrometer (Titertek Multiscan Plus MKII, Labsystems, Finland) after 60 min. The released amount of superoxide anion (O₂⁻) was deduced from the absorbance values at 550 nm (after subtraction of control values with SOD) using the cytochrome c extinction coefficient of 21×10³ M⁻¹ cm⁻¹ (See Massey, Biochim Biophys Acta, 34: 255(1959)). Results were expressed as nmoles O₂⁻ per 10⁶ cells per hour (FIG. 1).

C. Cytokine Release Assay

Cells (1×10⁶ PBM or 0.5×10⁶ AM/well) were distributed in 24-well plates (Falcon), preincubated in cRPMI for 24 hours at 37° C., 5% CO₂ with cytokines (IL-9, IL-4 (20 ng/ml) and IFN-γ (200 U/ml)) and stimulated for 20 hours with 1 μg/ml LPS without removing cytokines. Supernatants were harvested and frozen at −20° C. until cytokine titration. Release of TNF-α was quantified by a cytotoxicity bioassay using WEHI 164 cells clone 13, as previously described (Espevik and Nissen-Meyer, J. Immunol. Methods, 95:99-105 (1986) incorporated herein by reference), using rhTNF-α from Boehringer as a standard. IL-8, IL-10 and TGF-β1 concentrations were determined by ELISA. A kit from CLB (Amsterdam, The Netherlands) was used for IL-10 quantitation, following the manufacturer's protocol. A kit from Biosource allowed to determine TGF-β1 after the release from its latent complexes by a treatment of supernatants with ethanol acid (‘acid-treated supernatants’); TGF-P 1 was also assessed in crude supernatants (‘untreated supernatants’). To assay for IL-8, 96-well plates were coated overnight at 4° C. with 4 μg/ml anti-hIL-8 mAb (Sigma, clone 6217.11) in 100 mM sodium carbonate buffer (pH 9.6). After washings in PBS containing 0.1% v/v Tween 20 and blocking for 1 hour at 37° C. with 1% w/v BSA in the same buffer, rhIL-8 standards (Biosource International) and supernatants were incubated for 2 hour at 37° C. Plates were then incubated with 20 ng/ml biotinylated polyclonal anti-hIL-8 Ab (R§D Systems) in blocking buffer and, after washings, with HRP-conjugated streptavidin (Sigma). The reaction was then developed in 0.03% v/v H₂O₂ substrate and 0.42 mM 3,3′,5,5′-tetramethylbenzidine as chromogen in 100 mM sodium acetate/citric acid buffer (pH 4.9), stopped with 2 M H₂SO₄, and read in a plate spectrometer at 450 nm. The sensitivity of the TNF-α bioassay was 0.2 pg/ml, and that of IL-8, IL-10, and TGF-β1 immunoassays 10 pg/ml, 2 pg/ml, and 2 pg/ml, respectively. All supernatants were assayed in duplicate.

D. ERK½ Map Kinase Phosphorylation Assay

PBM (1×10⁶) were preincubated for 24 hour with medium alone or with cytokines (IL-9, IL-4 or TGF-β1 at 20 ng/ml) and stimulated from 5 min to 20 hour by 1 μg/ml LPS. When indicated, cells were pretreated for 1 hour with 100 pM PD98059 (a specific inhibitor of ERK½ phosphorylation, New England Biolabs, Beverly, Mass.), or for 15 min with 1 μM okadaic acid or 2.5 mM sodium orthovanadate as inhibitors of respectively serine/threonine (S/T) and tyrosine phosphatases (Sigma). PBM were lysed in ice-cold lysis buffer (20 mM Tris-HCl pH 7.4, 150 mM NaCl, 5 mM EDTA, 1% NP-40, 0.5% Na deoxycholate, 0.2% SDS) containing protease inhibitors (Roche Diagnostics) including freshly-added 1 mM PMSF, and protein phosphatase inhibitors (25 mM NaF, 1 mM Na₃VO₄) from Sigma. Cell extracts (10 μg, as determined by the bicinchoninic acid-based assay) were subjected to SDS-12%PAGE, and electrotransferred onto a nitrocellulose membrane (Amersham) immunoblotted to detect and compare phosphorylated (threonine²⁰²/tyrosine²⁰⁴ residues) and total ERK½, using specific antibodies and enhanced chemiluminescence (New England Biolabs). The results for pretreated and untreated cells were compared.

E. Immunofluorescence Staining

For FACS analysis, IL-9R expression on PBM and AM was assayed by indirect immunofluorescence. Adherent mononuclear cells (0.2×10⁶/well) were incubated at 4° C. for 1 hour with anti-hIL-9R mAb AH9R2 or AH9R7 diluted to 10 ug/ml in RPMI containing 3% v/v FBS. After three washings with RPMI-3%FBS, cells were incubated at 4° C. for 1 hour with 10 μg/ml SAM-FITC in the same medium. Cells incubated with mIgG2a or mIgG2b and thereafter with SAM-FITC, or only with SAM-FITC, served as negative controls for respectively primary and secondary Abs. After three washings, cells were fixed in 2% v/v formaldehyde in PBS-3%FBS for 15 min at room temperature, gently scraped with a rubber policeman and kept in the dark at 4° C. until FACS analysis performed on a FACscan from Becton Dickinson (Mountainview, Calif., USA).

Additional stainings for CD14 and Toll-like receptor (TLR)-4 were performed on PBM preincubated for 24 hour with cytokines, using respectively FITC-conjugated anti-CD14 mAb (clone MøP9, mIgG2b, Becton Dickinson) and anti-TLR4 rabbit Ab (Santa Cruz, Calif.) followed by F (ab′)2 fragments of mouse anti-rabbit IgG (MAR)-FITC.

Binding of IL-9 to the surface of PBM and AM was assessed by incubating these cells (0.2×10⁶) at 4° C. for 1 hour with hIL-9-mIgG3 chimeric molecule (10% COS cell supernatant). IL-9 binding was revealed after washings by incubation for 1 hour at 4° C. with FITC-conjugated goat anti-mouse IgG3 (GAM3-FITC, Southern Laboratories). Cells incubated with mIgG3 before GAM3-FITC served as the negative control. FACS analysis of the cell-associated fluorescence was then performed as for the assessment of IL-9R expression.

For confocal microscopy, mononuclear cells (0.2×10⁶/well) were cultured on coverslips for 2 hour in 24-well plates, washed with cRPMI, and immunostained for IL-9R as for FACS analysis with AH9R2 mAb. After washings with PBS-3% PBS and fixation by 2% formaldehyde in the same buffer, cells were mounted on slides with 2.5% 1,4-diacylbicyclo 2,2,2-octane (DABCO, Sigma) in Mowiol (Calbiochem-Novabiochem, Darmstadt, Germany), and analyzed by a MRC-1024 confocal microscope (Bio Rad Laboratories, Richmond, Calif., USA) using a 63X objective under oil immersion. Images were digitally recorded and reproduced with an ink-jet photo printer. Both for FACS and confocal microscopy, IL-9R negative and positive control cells consisted respectively in wild-type and hIL-9R-transfected Baf-3 cells (Demoulin et al., Mol. Cell Biol., 16: 4710-4716 (1996) incorporated herein by reference).

F. IL-9 Binding Assay

Binding of IL-9 to the surface of PBM and AM was assessed by incubating these cells (0.2×10⁶) at 4° C. for 1 hour with hIL-9-mIgG3 chimeric molecule (10%, v/v, COS cell supernatant). IL-9 binding was revealed after washings by incubation for 1 hour at 4° C. with GAM3-FITC. Cells incubated with mIgG3 before GAM3-FITC or only with GAM3-FITC served as negative controls. FACS analysis of the cell-associated fluorescence was then performed as for the assessment of IL-9R expression.

G. Preparation of IL-9 and Antibodies

Human IL-9 was purified from SF9 insect cell cultures infected with recombinant baculovirus by passage on Butyl Sepharose in 4 M NaCl buffer equilibrated to pH 7.5 with Tris-HCl buffer. The material eluted with 20 mM Tris-HCl pH 7.4 containing 1/10,000 v/v Tween 20 (Sigma) was further processed on Yellow3 Sepharose (Sigma) and eluted with 1 M NaCl in the same buffer. After dialysis against 50 mM acetate buffer pH 5.5, 1L-9 was adsorbed onto a Resource S cation exchange FPLC column and eluted with a NaCl gradient in the same buffer. Final polishing was performed by reversed-phase chromatography on a Vydac C4 column equilibrated in 0.05% trifluoroacetic acid and processed with a gradient of acetonitrile. Purity of this material was checked by silver-stained SDS-polyacrylamide gel electrophoresis.

An hIL-9-Ig fusion protein was produced as follows. The human IL-9 cDNA was amplified by PCR using a mutated antisense primer that introduced a BclI restriction site just before the stop codon: 5′-TCTTCTGATCATGCCTCT CATCCTCT-3′ SEQ ID NO: 1. The region comprising the hinge, CH2 and CH3 domains of the murine IgG3 isotype heavy chain was amplified by PCR using cDNA from the IgG3 anti-TNP hybridoma C3110 as a template with the following primers: 5′-AAGACTGAGTTGATCAAGAG AATCGAGCCTAGA-3′ (sense) SEQ ID NO: 2; 5′-AATGTCTAGATGCTGTTCT CATTTACC-3′ (antisense) SEQ ID NO: 3 containing BclI and Xbal sites for cloning. After amplification, both PCR products were digested with the appropriate restriction enzymes and cloned into the pcDNA/Amp plasmid (Invitrogen). Clones with the correct insert were transiently transfected into COS7 cells and supernatants were collected after 3 days.

H. Statistical Analysis

Data were obtained from experiments performed in triplicates and repeated at least three times, and results are expressed as means±SEM, except when indicated. The differences observed between the different groups were analyzed by the Student I test using InStat 2.01 statistical package (GraphPad InStat, San Diego, Calif.). P values less than 0.05 were considered significant.

The results presented herein demonstrate that monocytes/macrophages are targets for IL-9. The results demonstrate that the inhibition by IL-9 on oxidative burst in monocytes/macrophages are achieved by a pretreatment period, preferably the monocytes are contacted with IL-9 at least about 24 hours before the cells are contacted with an agent that stimulates the monocytes/macrophages. The monocytes/macrophages may be treated with the IL-9 between about 24 hours and about 96 hours before contacting the cells with an agent that stimulates the monocytes/macrophages. IFN-γ does not antagonize the effects of IL-9 but this is not due to a down regulation of the IFN-γR by IL-9. In addition, the effects of IL-9 and IL-4 on PMA-induced oxidative burst in monocytes are additive. These results suggest that IL-4 and IL-9 use different mechanisms to mediate their effects on monocytes/macrophages.

IL-9 does not down regulate CD14 expression, which is in contrast to the effect of IL-4 on CD 14 expression, and indicates that IL-9 does not function through CD 14 to inhibit oxidative burst or regulate cytokine release. IL-10 is a major monocyte-macrophage suppressing factor that inhibits the production of inflammatory mediators such as TNF-α and ROI, but IL-9 appears to inhibit oxidative burst and regulate cytokine release through an IL-10 independent mechanism. In contrast, monoclonal antibodies to TGF-P1 significantly reduced IL-9's inhibition of oxidative burst in LPS-stimulated PBM and AM. In addition, TGF-β downregulated oxidative burst in LPS stimulated monocytes. These results indicate that IL-9 induces TGF-β in LPS-activated monocytes and macrophages, and TGF-β mediates, at least partly, the effects of IL-9 on oxidative burst.

Stimulation of the growth and/or activation state of Th2 lymphocytes and mast cells, as well as induction of hypereosinophilia, are thought to explain both beneficial and deleterious activities of IL-9 in Th2-related disorders, such as parasitic infections or asthma. The present finding that mononuclear phagocytes are regulated by IL-9 may be more specifically relevant to inflammatory disorders, such as the inflammatory bowel diseases, e.g. Crohn's disease and rectocolitis, and other tissue injury resulting from an exaggerated inflammatory response, which includes the uncontrolled release of ROI and sepsis, in which monocyte/macrophage activation plays a central role. Exaggerated inflammatory responses may lead to e.g. liver damage by toxic substances (e.g., alcohol or carbon tetratrachloride), acute lung damage and ARDS, encephalomyelitis and brain injury following ischemia, and damage to articular (joint) tissue, as in rheumatoid arthritis. Interestingly, it was recently shown that administration of IL-9 prevented mortality in mice challenged with Pseudomonas aeruginosa but not in those challenged with LPS. (Grohmann et al., J. Immunol. 164:4197-4203 (2000)). This beneficial effect was dependent on a prophylactic administration of IL-9 since no improvement in survival was observed when rIL-9 was injected concomittantly or after the infectious challenge. In this model, IL-9 treatment was associated with the suppression of serum TNF-α, as well as IL-12/P40 and IFN-γ. However, in contrast with TNF-α, which is reduced by IL-9 both in the model reported in Grohmann et al., 2000 (supra) and in our study, IL-10 was up regulated in serum from IL-9-treated mice challenged with LPS. This apparent discrepancy between the endotoxemia in vivo model and the results presented herein might be due to the fact that the main source of IL-10 in mice treated with IL-9 was possibly the lymphocyte population because induction of IL-10 expression was observed in the spleen regardless of the particular cell type. Thus, regulation of IL-10 production by IL-9 might differ between lymphocytes and monocytes. The protection of mice from lethal endotoxemia was shown with other Th2 cytokines, e.g. IL-4, IL-10 and IL-13 (Grohmann et al., J. Immunol. 164:4197-4203 (2000); Giampietri et al., Cytokine, 12:417-421 (2000); Howard et al., J. Exp. Med., 177:1205-1208 (1993); Gerard et al., J. Exp. Med., 177:547-550 (1993) and; Muchamuel et al., J. Immunol., 158:2898-2903 (1997)), and was associated with a reduction of TNF-α production. Th2 cytokines-mediated protection in in vivo models of exaggerated inflammatory response may be related to the capacity observed in vitro of these cytokines to inhibit stimulation of mononuclear phagocytes.

In alveolar macrophages, which represent a mature tissular mononuclear phagocyte, IL-9 inhibited LPS-stimulated oxidative burst to a similar extent as that observed in blood monocyte, as previously reported for IL-4 (Bhaskaran et al., J. Leukoc. Biol., 52:218-223 (1992)). In contrast, macrophage TNF-α and IL-10 release was not significantly modulated by IL-9. Without wishing to be bound by theory this apparent loss of IL-9 activity on cytokine release by tissue macrophages as compared to blood monocytes might result from the reduced expression of IL-2R γ chain (a receptor subunit also shared by IL-9R and IL-4R) observed during monocyte differentiation, as was proposed to explain the loss of IL-4 activity on macrophages by Hart et al., (Bonder et al., Immunol., 96:529-536 (1999) and Hart et al., J. Leukoc. Biol., 66:575-578 (1999)).

The results presented herein demonstrate that IL-9 pretreatment inhibits the oxidative burst in activated mononuclear phagocytes, e.g., blood monocytes and alveolar macrophages. Without wishing to be bound by theory, the mechanism of this inhibition may involve IL-9 induction of TGF-β secretion by activated monocytes/macrophages which, in turn, inhibits their oxidative burst through ERK inactivation. IL-9 also suppresses the TNF-α release by blood monocytes, but not by alveolar macrophages. These findings highlight monocytes/macrophages as target cells for IL-9, and suggest that monocyte/macrophage deactivation by IL-9 may be of crucial importance in maintaining host tissue integrity during inflammatory processes.

We claim:

1. A method for treating a subject having a pathologic disorder associated with stimulated mononuclear phagocytes, or a subject at risk for developing the pathologic disorder, comprising administering an effective amount of IL-9, or a portion of IL-9 sufficient to bind to IL-9 receptors, to said subject wherein the effective amount of IL-9 is sufficient to inhibit the stimulation of mononuclear phagocytes.

2. The method of claim 1, wherein the pathologic disorder is selected from the group consisting of sepsis, atherosclerosis, pancreatitis, gastric ulcer, small intestine ischemia, liver tissue injury, lung tissue injury, central nervous tissue injury and arthritis.

3. The method of claim 1 wherein the pathologic disorder is selected from the group consisting of acute respiratory distress syndrome and an allergic inflammatory disorder of the bowel.

4. The method of claim 3 wherein the inflammatory disorder of the bowel is selected from the group consisting of ulcerative colitis and Crohn's disease.

5. The method of claim 1 wherein the IL-9 or portion of IL-9 is administered prior to contacting said mononuclear phagocytes with an agent that stimulates mononuclear phagocytes.

6. The method of claim 1 wherein the IL-9 or portion of IL-9 is administered for at least 24 hours prior to contacting said mononuclear phagocytes with an agent that stimulates mononuclear phagocytes.

7. The method of claim 1, wherein said mononuclear phagocytes are peripheral blood monocytes or alveolar macrophages.

8. The method of claim 1, wherein said agent that stimulates mononuclear phagocytes stimulates production of a reactive oxygen intermediates by said stimulated mononuclear phagocytes.

9. The method of claim 8, wherein the reactive oxygen intermediate is selected from the group consisting of H₂O₂ and O₂⁻.

10. The method of claim 1, wherein the agent that stimulates the mononuclear phagocytes is selected from the group consisting of a cytokine, a viral coat, a bacterial component, a lipoteichoic acid (LTA), an FcR triggering agent, phorbol myristate acetate (PMA), an alcohol, carbon tetrachloride and a hemodialysis membrane.

11. The method of claim 10, wherein the bacterial component is a component of a gram positive bacterium

12. The method of claim 10, wherein the bacterial component is a component of a gram negative bacterium.

13. The method of claim 10, wherein the bacterial component is selected from the group consisting of a cell membrane, an enzyme, an endotoxin and lippopolysaccharide (LPS).

14. The method of claim 10, wherein the Fc receptor-triggering agent is an antigen-antibody complex or a phagocytosed particle.

15. The method of claim 14, wherein the phagocytosed particle is opsonized zymosan.

16. The method of claim 10, wherein the cytokine is IFN-γ and TNF-α.

17. The method of claim 1, wherein the subject at risk for developing a pathologic disorder is an immunocompromised subject.

18. The method of claim 1, wherein the subject at risk for developing a pathologic disorder is a subject undergoing, or will undergo, a medical procedure.

19. The method of claim 18, wherein the medical procedure is selected from the group consisting of chemotherapy, radiation therapy, immunotherapy, immunization, transfusion, a transplantation, infusion, reperfusion, hemodialysis and ischemia reperfusion.

20. The method of claim 1, wherein the IL-9 is administered to the subject about 24 to about 96 hours prior to contact with said agent that stimulates mononuclear phagocytes.

21. The method of claim 1, wherein the IL-9 is administered to the subject at least 24 hours prior to contact with said agent that stimulates mononuclear phagocytes.

22. A method to inhibit the production of TNF-α by stimulated peripheral blood monocytes (PBM) comprising contacting a sample containing PBM with an effective amount of IL-9 prior to contacting the sample containing PBM with an agent that stimulates the production of TNF-α from the PBM, wherein the sample is contacted with IL-9 for sufficient time to inhibit production of TNF-α from the PBM.

23. The method of claim 22, wherein the agent that stimulates release of TNF-α is selected from the group consisting of a cytokine, a viral coat, a bacterial component, a membrane, an enzyme, lipopolyssacharide (LPS), lipoteichoic acid (LTA), an FcR triggering agent, a phagocytosed particle, phorbol myristate acetate (PMA), an alcohol, carbon tetrachloride and a hemodialysis membrane.

24. The method of claim 23, wherein the bacterial component is a component of a gram positive bacterium.

25. The method of claim 23, wherein the bacterial component is a component of a gram negative bacterium.

26. The method of claim 23, wherein the bacterial component is selected from the group consisting of a cell membrane, an enzyme, an endotoxin and lippopolysaccharide (LPS).

27. The method of claim 23, wherein the Fc receptor-triggering agent is an antigen-antibody complex or a phagocytosed particle.

28. The method of claim 27, wherein the phagocytosed particle is opsonized zymosan.

29. The method of claim 23, wherein the cytokine is IFN-γ and TNF-α.

30. The method of claim 22, wherein the IL-9 is administered to the subject at least 24 hours prior to contact with said agent that stimulates the production of TNF-α.

31. The method of claim 30, wherein the IL-9 is administered to the subject at least 24 hours to about 96 hours prior to contact with said agent that stimulates the production of TNF-α.

32. A method for potentiating the production of TGF-β from mononuclear phagocytes comprising contacting a sample containing mononuclear phagocytes with an effective amount of IL-9 and then contacting the mononuclear phagocytes with an agent that stimulates the production of TGF-β from the mononuclear phagocytes wherein the sample is contacted with the IL-9 for sufficient time to promote production of TGF-β from the stimulated mononuclear phagocytes.

33. The method of claims 32, wherein said mononuclear phagocytes are peripheral blood monocytes or alveolar macrophages.

34. The method of claim 32, wherein the agent that stimulates the production of TGF-β is selected from the group consisting of a cytokine, a viral coat, a bacterial component, a membrane, an enzyme, lipopolyssacharide (LPS), lipoteichoic acid (LTA), an FcR triggering agents, a phagocytosed particle, phorbol myristate acetate (PMA), an alcohol, carbon tetrachloride, and a hemodialysis membrane.

35. The method of claim 32, wherein the bacterial component is a component of a gram positive bacterium.

36. The method of claim 32, wherein the bacterial component is a component of a gram negative bacterium.

37. The method of claim 32, wherein the bacterial component is selected from the group consisting of a cell membrane, an enzyme, an endotoxin and lippopolysaccharide (LPS).

38. The method of claim 32, wherein the Fc receptor-triggering agent is an antigen-antibody complex or a phagocytosed particle.

39. The method of claim 32, wherein the phagocytosed particle is opsonized zymosan.

40. The method of claim 32, wherein the IL-9 is administered to the subject at least 24 hours prior to contact with said agent that stimulates the production of TGF-β.

41. The method of claim 32, wherein the IL-9 is administered to the subject at least 24 hours to about 96 hours prior to contact with said agent that stimulates the production of TGF-β.

42. A method for inhibiting oxidative burst in mononuclear phagocytes by inhibiting the activation of extracellular signal-regulated kinase (ERK) in said mononuclear phagocytes said method comprising contacting a sample containing mononuclear phagocytes with IL-9 prior to contacting said sample containing mononuclear phagocytes with an agent that promotes phosphorylation of ERK, wherein the cells are contacted with IL-9 for a sufficient time to inhibit the activation of ERK.

43. The method of claims 42, wherein said mononuclear phagocytes are peripheral blood monocytes or alveolar macrophages.

44. The method of claim 42, wherein the agent that promotes phosphorylation of ERK is selected from the group consisting of a cytokine, a viral coat, a bacterial component, a membrane, an enzyme, lipopolyssacharide (LPS), lipoteichoic acid (LTA), an FcR triggering agent, a phagocytosed particle, phorbol myristate acetate (PMA), an alcohol, carbon tetrachloride, and a hemodialysis membrane.

45. The method of claim 44, wherein the bacterial component is a component of a gram positive bacterium.

46. The method of claim 44, wherein the bacterial component is a component of a gram negative bacterium.

47. The method of claim 44, wherein the bacterial component is selected from the group consisting of a cell membrane, an enzyme, an endotoxin and lippopolysaccharide (LPS).

48. The method of claim 44, wherein the Fc receptor-triggering agent is an antigen-antibody complex or a phagocytosed particle.

49. The method of claim 46, wherein the phagocytosed particle is opsonized zymosan.

50. The method of claim 44, wherein the cytokine is IFN-γ and TNF-α.

51. The method of claim 42, wherein the IL-9 is administered to the subject at least 24 hours prior to contact with said agent that promotes phosphorylation of ERK.

52. The method of claim 41, wherein the IL-9 is administered to the subject at least 24 hours to about 96 hours prior to contact with said agent that promotes phosphorylation of ERK.

53. A method for antagonizing the priming effect of IFN-γ on the stimulation of mononuclear phagocytes comprising contacting a sample containing mononuclear phagocytes that are primed with IFN-γ with an effective amount of IL-9 at least 24 hours prior to treating said primed mononuclear phagocytes with an agent that stimulates mononuclear phagocytes.

54. A method for inhibiting the production of IL-10 by stimulated peripheral blood monocytes (PBM) comprising contacting said PBM with an effective amount of IL-9 for a sufficient duration prior to contacting the PBM with an agent that stimulates the PBM wherein said effective amount of IL-9 is an amount sufficient to inhibit IL-10 production by PBM as compared to PBM that were not contacted with IL-9.
